(19) United States
(12) Patent Application Publication
(10) Pub. No.: US 2008/0065082 A1
(43) Pub. Date: Mar. 13, 2008

(54) STEERABLE RASP/TRIAL INSERTER

(76) Inventors: Narissa Chang, Mansfield, MA (US); Justin Dye, Mansfield, MA (US)

Correspondence Address: CARR LLP (IST), 670 FOUNDERS SQUARE, 900 JACKSON STREET, DALLAS, TX, 75202, US

(21) Appl. No.: 11/852,183

(22) Filed: Sep. 7, 2007

Related U.S. Application Data
(60) Provisional application No. 60/825,091, filed on Sep. 8, 2006.
(60) Provisional application No. 60/868,022, filed on Nov. 30, 2006.

(51) Int. Cl. A61B 17/00; A61B 17/58

(52) U.S. Cl. 606/85; 606/99

(57) ABSTRACT

Instruments and methods are provided for inserting a rasp into an intervertebral space of a spine and using the rasp to decorticate the adjacent vertebra. More particularly, one embodiment provides an instrument that actively changes the angle of the rasp relative to the instrument. The delivery instrument may use a gear portion to articulate the rasp. A second gear on the rasp may mate with a corresponding gear on the instrument. As the instrument gear rotates relative to the instrument, the instrument gear drives the rasp gear, thereby rotating the rasp to decorticate the vertebra. Trial inserts and methods are also provided to determine an appropriate size of a rasp for decortication.

### CROSS-REFERENCE TO RELATED APPLICATIONS

This application relates to, and claims the benefit of the filing date of, co-pending U.S. provisional patent application Ser. No. 60/528,091 entitled “Steerable Rasp/Trial Inserter” filed Sep. 8, 2006; co-pending U.S. provisional patent application Ser. No. 60/826,716 entitled “Steerable Rasp/Trial Inserter and Method of Use” filed Sep. 22, 2006; and co-pending U.S. provisional patent application Ser. No. 60/868,022 entitled “Steerable Rasp/Trial Inserter” filed Nov. 30, 2006; the entire contents of which are incorporated herein by reference for all purposes.

### TECHNICAL FIELD

The invention relates generally to instruments and methods for spinal surgery and, more particularly, to instruments for inserting and positioning interbody devices or spacers in the intervertebral space of a spine.

### BACKGROUND

A spine such as a human spine is a complex structure designed to achieve a myriad of tasks, many of them of a complex kinematic nature. The spinal vertebrae allows the spine to flex in three axes of movement relative to the portion of the spine in motion. These axes include the horizontal (i.e., bending either forward/anterior or aft/posterior), roll (i.e., lateral bending to either left or right side) and rotation (i.e., twisting of the shoulders relative to the pelvis).

The intervertebral spacing (e.g., between neighboring vertebrae) in a healthy spine is maintained by a compressible and somewhat elastic disc. The disc serves to allow the spine to move about the various axes of rotation and through the various arcs and movements required for normal mobility. The elasticity of the disc maintains spacing or distance between the vertebrae, allowing room or clearance for compression of neighboring vertebrae during flexion and lateral bending of the spine. In addition, the disc allows relative rotation about the vertical axis of neighboring vertebrae, permitting twisting of the shoulders relative to the hips and pelvis. The clearance between neighboring vertebrae maintained by a healthy disc is also important to allow the nerves from the spinal cord to extend out of the spine, between neighboring vertebrae, without being squeezed or impinged by the vertebrae.

In situations (e.g., based upon injury or otherwise) where a disc is not functioning properly, the inter-vertebral disc tends to compress, and in doing so pressure is exerted on nerves extending from the spinal cord by this reduced inter-vertebral spacing. Various other types of nerve problems may be experienced in the spine, such as exiting nerve root compression in neural foramen, passing nerve root compression, and enervated annulus (i.e., where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to each other by surgically removing an improperly functioning disc and replacing it with a lumbar interbody fusion (LIF) device or spacer. Although prior interbody devices, including LIF cage devices, can be effective at improving patient condition, the vertebrae of the spine, body organs, the spinal cord, other nerves, and other adjacent bodily structures make it difficult to obtain surgical access to the locations between the vertebrae in which the LIF cage is to be installed.

Generally speaking, the surfaces of the vertebrae adjacent to the spacer need to be decorticated prior to inserting the spacer within the intervertebral space. The decortication leaves the end surfaces of the vertebrae hemorrhaging, thereby promoting bone growth from the vertebrae. Subsequently, the growing bone envelopes the spacer and fuses the adjacent vertebrae together. However, the geometry of the vertebrae and surrounding tissue makes it difficult to insert decortication instruments into the intervertebral space. For similar reasons, moving the decortication instruments (e.g., to clean the boney material) is also difficult. What is needed, therefore, are instruments for decorticating vertebrae in a minimally invasive manner.

### SUMMARY

Instruments and methods are provided for inserting a removable insert into the intervertebral space of a human spine. More particularly, one embodiment provides a surgical instrument that actively changes the angle of a removable insert relative to the surgical instrument via a drive member. The surgical instrument of the embodiment may use gear teeth to articulate the removable insert. A plurality of gear teeth on the removable insert may mate with corresponding protrusions provided on the drive member of the surgical instrument.

A method of traumatizing a pair of adjacent vertebral endplates is provided by another embodiment that comprises placing a leading end of a distal insert coupled to a surgical instrument in a first position between two adjacent vertebral endplates. The method further comprises moving the distal insert to a second position between the adjacent vertebral endplates by impacting the proximal end portion of the surgical instrument and pivoting the distal insert to a second angular position relative to the body by rotating the handle about the body. The method also comprises locking the second angular position of the distal insert and moving the distal insert to a third position between the adjacent vertebral endplates by impacting the proximal end portion of the surgical instrument.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1A is an oblique perspective view of an instrument constructed in accordance with an embodiment of the present invention;

FIG. 1B is a side elevation view of the instrument of FIG. 1A with an attached rasp in a rotated position;

FIG. 1C is a side elevation view of the instrument of FIG. 1A with an attached rasp in a straight position;

FIG. 1D is a top view of the instrument of FIG. 1A;

FIG. 1E is a cross-sectional view of the rasp of FIG. 1B as seen along line 1E-1E;

FIG. 1F is a side elevation view of another instrument constructed in accordance with an embodiment of the present invention;

FIG. 2A is a cross-sectional view of the instrument of FIG. 1A as seen along the line 2A-2A, illustrating the rasp in a straight position;

FIG. 2B is a cross-sectional detail view of a distal end of the instrument and the rasp of FIG. 2A, illustrating the rasp in a straight position;

FIG. 2C is a cross-sectional detail view of a knob at a proximal end of the instrument of FIG. 2A, illustrating the position of the knob when the rasp is in a straight position;

FIG. 2D is a cross-sectional view of the instrument of FIG. 1B as seen along the line 2D-2D, illustrating the rasp in a rotated position;

FIG. 2E is a cross-sectional detail view of a distal end of the instrument and the rasp of FIG. 2D, illustrating the rasp in a rotated position;

FIG. 2F is a cross-sectional detail view of a knob at a proximal end of the instrument of FIG. 2D, illustrating the position of the knob when the rasp is in a rotated position;

FIG. 3A is an oblique perspective view of the rasp of FIG. 1A;

FIG. 3B is another oblique perspective view of the rasp of FIG. 3A;

FIG. 3C is a side elevation view of the rasp of FIG. 3A;

FIG. 3D is a top view of the rasp of FIG. 3A;

FIG. 3E is another side elevation view of the rasp of FIG. 3A;

FIG. 3F is an end elevation view of the rasp of FIG. 3A;

FIG. 3G is a perspective view of a distal end of an embodiment of an instrument at which location the rasp of FIG. 3A connects to the instrument;

FIG. 4A is an oblique perspective view of an instrument constructed in accordance with an embodiment of the present invention;

FIG. 4B is a side elevation view of the instrument of FIG. 4A with a trial insert in a rotated position;

FIG. 4C is another side elevation view of the instrument of FIG. 4A with a trial insert in a straight position;

FIG. 4D is a top view of the instrument of FIG. 4A;

FIG. 4E is a cross-sectional view of the trial insert of FIG. 4B as seen along line 4E-4E;

FIG. 5 is a side elevation view of the instrument of FIG. 1A with a rasp positioned in an intervertebral space;

FIG. 6A is a side elevation view of an instrument constructed in accordance with an embodiment of the present invention with a rasp in a rotated position;

FIG. 6B is a top view of the instrument of FIG. 6A;

FIG. 6C is a cross-sectional view of the instrument of FIG. 6B as seen along line 6C-6C, with the rasp in a rotated position;

FIG. 6D is a cross-sectional detail view of a distal end of the instrument and rasp of FIG. 6C, illustrating the rasp in a rotated position;

FIG. 6E is a cross-sectional detail view of a knob at a proximal end of the instrument of FIG. 6C, illustrating the position of the knob when the rasp is in a rotated position;

FIG. 7A is a side elevation view of the instrument of FIG. 6 with the rasp in a straight position;

FIG. 7B is a top view of the instrument of FIG. 7A;

FIG. 7C is a cross-sectional view of the instrument of FIG. 7B as seen along line 7C-7C, with the rasp in a straight position;

FIG. 7D is a cross-sectional detail view of a distal end of the instrument and rasp of FIG. 7C, illustrating the rasp in a straight position;

FIG. 7E is a cross-sectional detail view of a knob at a proximal end of the instrument of FIG. 7C, illustrating the position of the knob when the rasp is in a straight position;

FIG. 8A is an oblique perspective view of the rasp of FIG. 6;

FIG. 8B is a top plan view of the rasp of FIG. 6;

FIG. 8C is a side elevation view of the rasp of FIG. 6;

FIG. 8D is an end elevation view of the rasp of FIG. 6;

FIG. 9A is an oblique perspective view of the instrument of FIG. 6 with a trial insert in a rotated position;

FIG. 9B is bottom view of the instrument of FIG. 9A;

FIG. 9C is side elevation view of the instrument of FIG. 9A;

FIG. 9D is top plan view of the instrument of FIG. 9A;

FIG. 10 is a side elevation view of the instrument of FIG. 6 with the rasp in an intervertebral space;

FIG. 11 is an illustrative embodiment of a kit comprising the surgical instrument of FIG. 1A;

FIG. 12 is an illustrative embodiment of a kit comprising the surgical instrument of FIG. 6A.

### DETAILED DESCRIPTION

The entire contents of the following provisional patent applications are incorporated herein by reference for all purposes: U.S. provisional patent application Ser. No. 60/528,091 entitled “Steerable Rasp/Trial Inserter” filed Sep. 8, 2006; co-pending U.S. provisional patent application Ser. No. 60/826,716 entitled “Steerable Rasp/Trial Inserter and Method of Use” filed Sep. 22, 2006; and co-pending U.S. provisional patent application Ser. No. 60/868,022 entitled “Steerable Rasp/Trial Inserter” filed Nov. 30, 2006

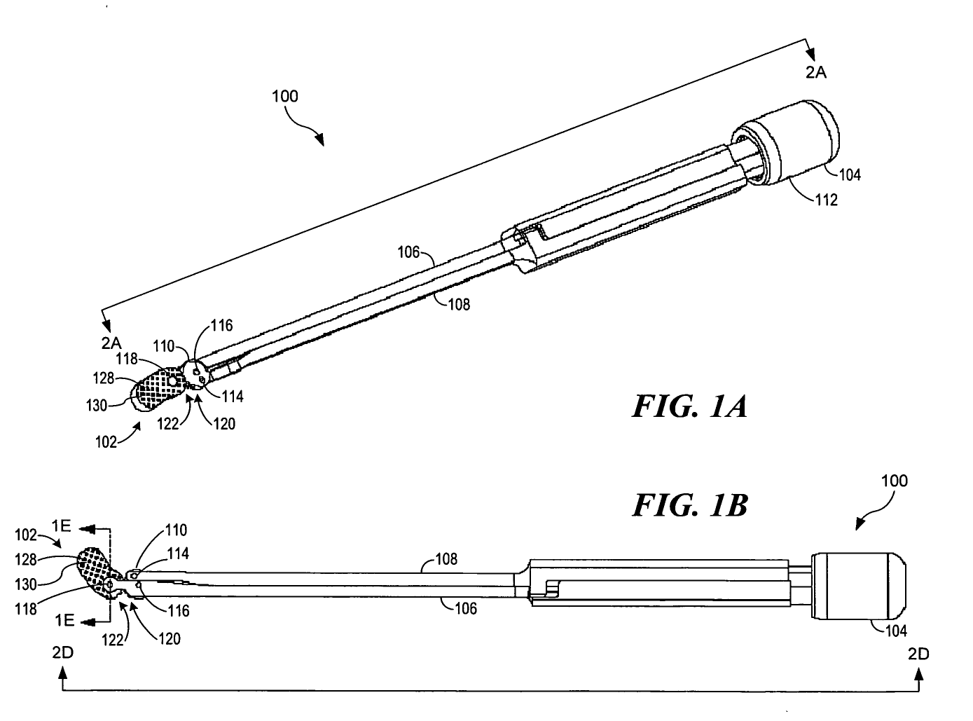

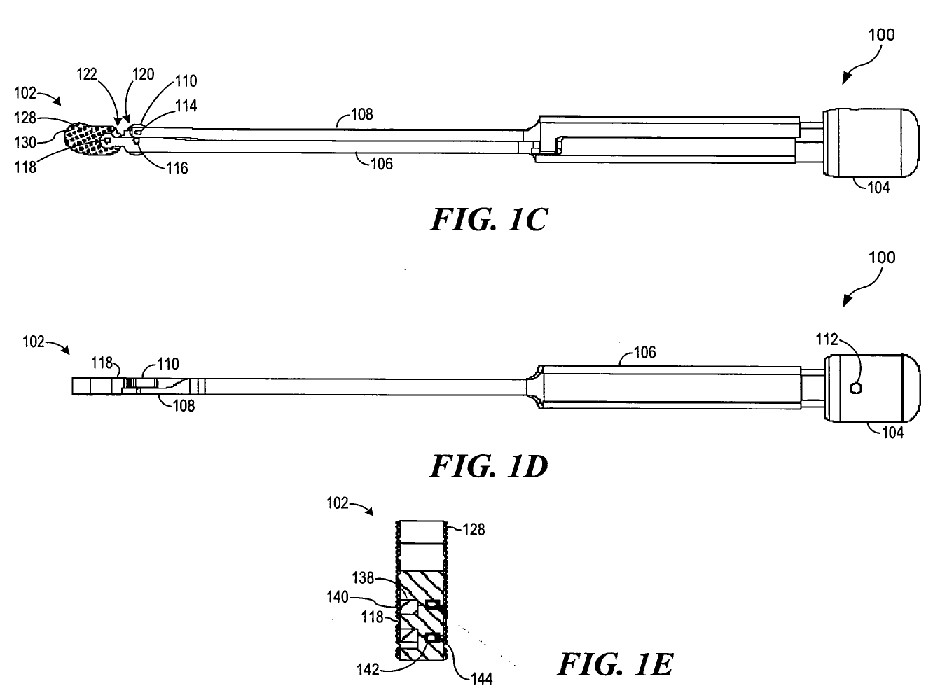

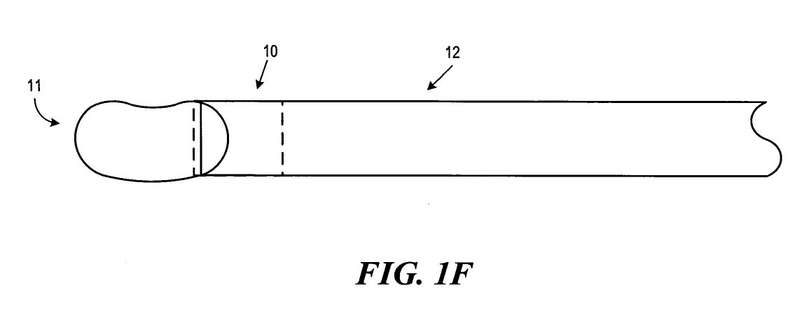

Referring to FIGS. 1A-1F of the drawings, the reference numeral 100 generally designates an instrument embodying features of an aspect of the present invention. The instrument 100 comprises features that may enable a rasp 102 to be attached to the instrument 100, inserted into an intervertebral space, rotated therein for decorticating or traumatizing the adjacent vertebral endplates, and withdrawn from the space. More particularly, the instrument 100 may comprise an articulation knob 104 (e.g., an actuating mechanism), a main body 106 (e.g., a guide member), an articulation bar 108 (e.g., an elongated member), and a driving gear 110. Additionally, the instrument 100 may comprise several pins 112, 114, 116, and 118, for example.

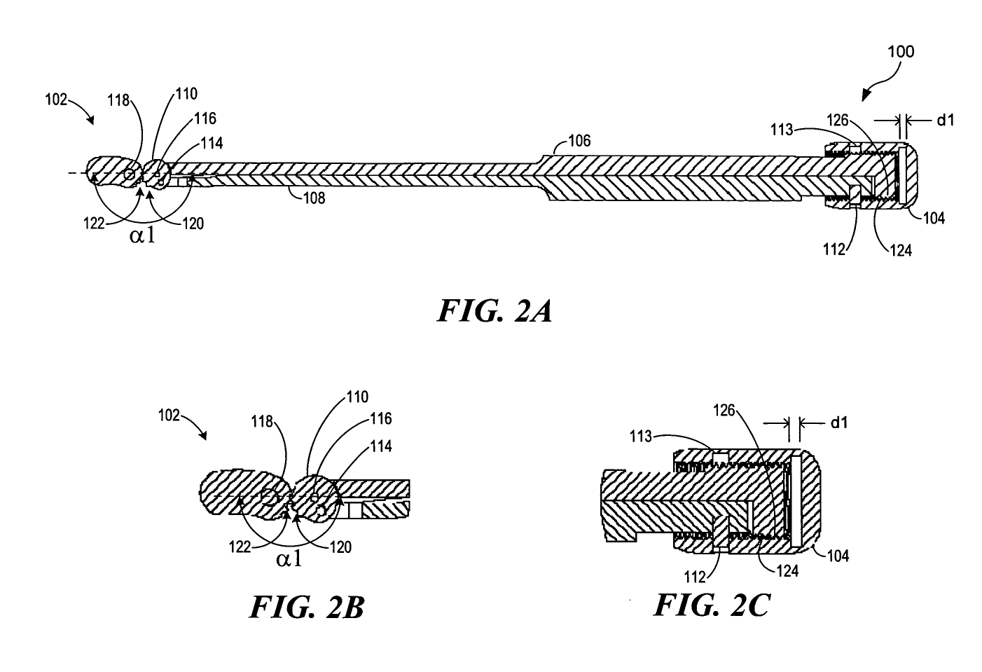

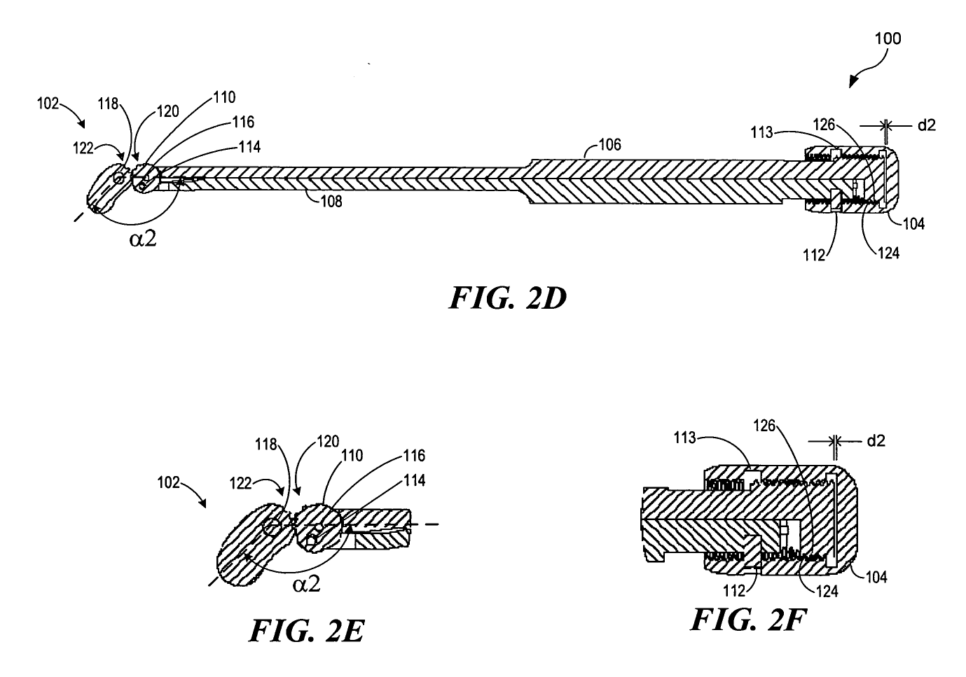

Pin 112 may attach the knob 104 to the articulation bar 108 so that the two objects can translate together along the main body 106. Further, the pin 112 may travel or slide along a groove 113 (see FIGS. 2A, 2C, 2D, and 2F), located on the interior of the knob 104, as the knob 104 is rotated and the articulation bar 108 is translated. In some embodiments, pin 114 pivotally attaches the articulation bar 108 to the driving gear 110 at a distal end of the articulation bar 108. Similarly, pin 116 may pivotally attach the driving gear 110 to the main body 106. In particular, pins 114 and 116 may be offset from each other in such a manner that when the articulation bar 108 translates, the pinned connection at pin 114 rotates the gear 110 about the pin 116. Because the driving gear 110 may comprise a set of gear teeth 120, which may mesh with a corresponding set of gear teeth 122 on the rasp 102, rotation of the driving gear 110 may also rotate the rasp 102 about the pinned connection at pin 118, located between the rasp 102 and the main body 106 (see FIGS. 2A, 2B, 2D, and 2E).

Furthermore, the knob 104 and main body 106 may each comprise corresponding threaded sections 124 and 126. As a result, when the articulation knob 104 is rotated, the threaded sections 124 and 126 may cause the knob 104 to translate along the main body 106. In turn, the translation of the knob 104 may also result in the translation of the articulation bar 108, thereby rotating the driving gear 110. The driving gear 110, in turn, may rotate the rasp 102. Consequently, the rotation of the knob 104 may cause the rasp 102 to rotate.

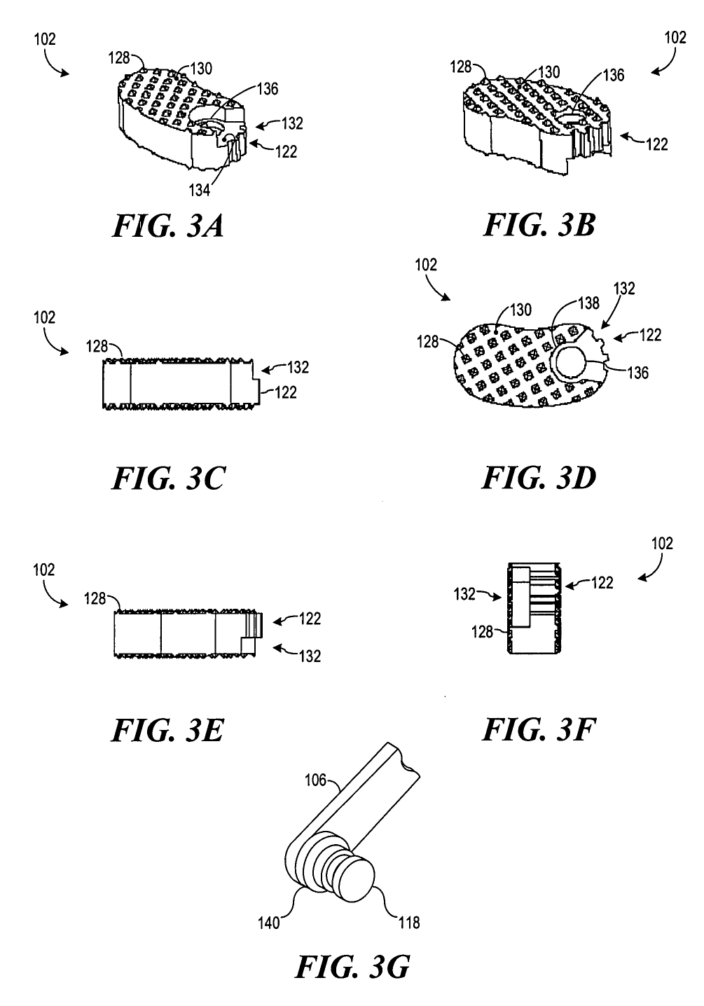

The rasp 102 may generally comprise a relatively large number of protruding teeth 128, spread across both of the top and bottom surfaces 130 of the rasp 102 (see FIGS. 3A and 3B). These protruding teeth 128 may allow the rasp 102 to decorticate (i.e., clean or scrape) the end plates (i.e., the ends or surfaces) of the adjacent vertebra as the rasp 102 is rotated within an intervertebral space. To enable the rasp 102 to rotate and be removably attached to the main body 106, the rasp 102 may comprise some of the features illustrated by FIGS. 1E, and 3A-3F. For example, the rasp 102 may comprise an attachment area 132 defined by a landing 134. A hole 136 may be located in the attachment area 132 within the landing 134, spaced apart from the geared end of the rasp 102. The hole 136 may accept the pin 118 so that the rasp 102 may rotate about the pin 118 while the attachment area landing 134 provides clearance between the main body portion 106 and the body of the rasp 102. Consequently, the landing 134 may enable the rasp 102 to attach to the instrument 100 without adding to the overall height of the rasp 102 and instrument 100 assembly.

The landing 134 may also define a raceway 138 located between the hole 136 and the protruding teeth 128 covered surfaces 130. A generally annular guide 140 (see FIG. 3G) of the main body 106 may slidably engage the raceway 138. Alternatively, the main body 106 (without a guide 140) may rest flush against the landing 134. Another raceway 142 within the rasp 102 may accommodate a coil spring 144. The coil spring 144 may be biased to press against the pin 118, which connects the rasp 102 to the main body 106. As a result, friction between the coil spring 144 and the pin 118 may hold the rasp 102 adjacent to the instrument 100.

With reference now to FIGS. 2A-2F, an operation of an embodiment of the present invention is depicted using the instrument 100 and rasp 102 for example. More particularly, FIGS. 2A and 2B show the rasp 102 in a relatively straight position designated by the angle “α1.” While FIGS. 2D and 2E show the rasp 102 in a rotated position indicated by the angle “α2.” In operation (and assuming that the rasp 102 may have been initially attached to the instrument 100 while the instrument 100 was configured to orient the rasp 102 in a straight position), the articulation knob 104 may be located such that an internal surface of an end of the knob 104 is a distance “d1” (see FIG. 2C) away from a proximal end of the main body 106. As a user rotates the knob 104 toward the main body 106, the knob 104 moves towards a position in which the end of the knob 104 and the proximal end of the main body 106 is a distance “d2” (see FIG. 2F) apart. The pin 112 that connects the knob 104 to the articulation bar 108 may cause the articulation bar 108 to translate through a corresponding distance. The articulation bar 108 consequently pushes against the pin 114, thereby causing the rotation of the driving gear 110. With the two sets of gear teeth 120 and 122 meshed (or engaged) between the driving gear 110 and the rasp 102, the rasp 102 may be rotated between an angle “α1” and an angle “α2” (i.e., between the straight and the rotated positions).

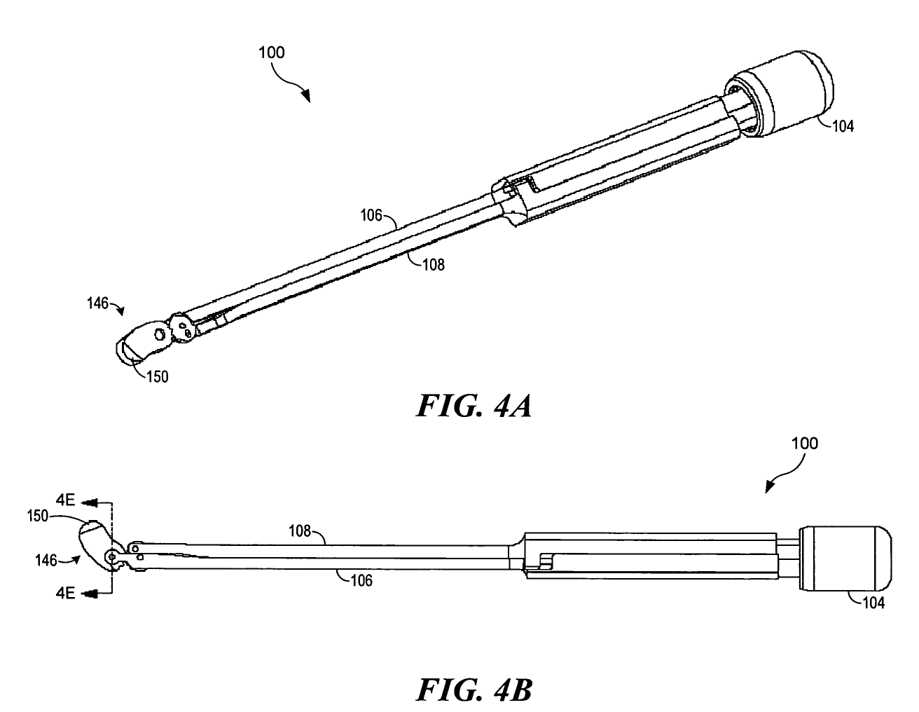

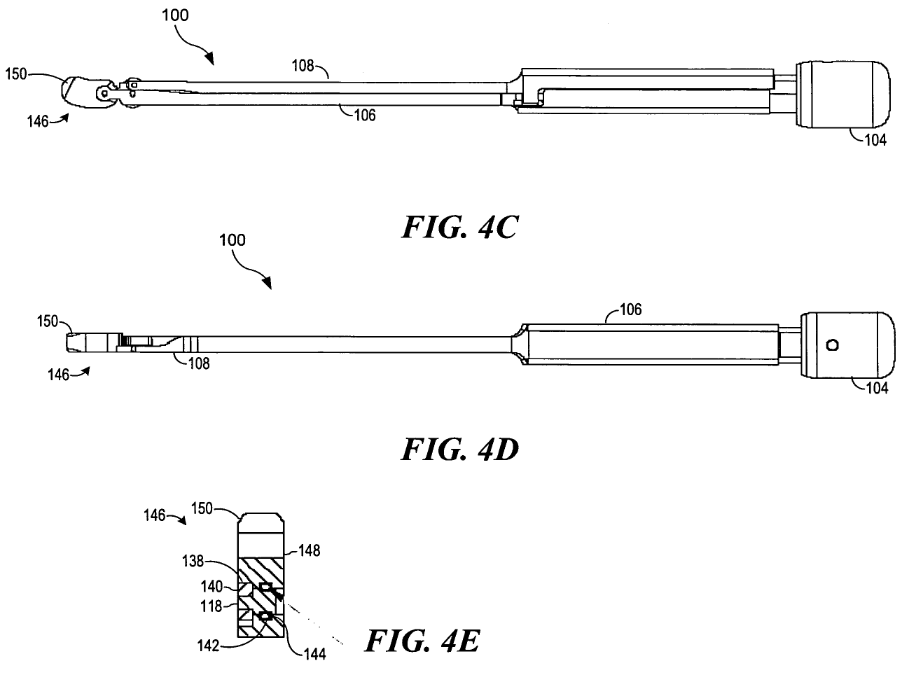

FIGS. 4A-4E illustrate another embodiment of the present invention. In some embodiments, such as the one shown in FIGS. 4A-4E, a trial insert 146 may be connected to the instrument 100 instead of a rasp 102. Generally, the trial insert 146 is similar to the rasp 102 and may attach to the instrument 100 in much the same way as did the rasp 102. However, the trial insert 146 may also differ from the rasp 102 in several ways. For example, various trial inserts 146 can be provided, each comprising a different surface-to-surface thickness. A user can therefore insert various thicknesses of trial inserts 146 into the intervertebral space until the user is able to determine an appropriate size for rasp 102 (which corresponds to the various sizes of trial inserts 146) that the user may desire to use in order to decorticate the vertebrae. Therefore, the trial inserts 146 do not need to comprise protruding teeth on their surfaces (or faces) 148. In addition, the trial inserts 146 may also comprise an angled landing 150 on a distal end so that when the trial insert 146 is inserted between the vertebrae, the trial insert 146 may impart less force on the vertebra than would otherwise be the case. This feature (i.e., the angled landing 150) may be useful for some situations (among others) in which the user is able to determine that the current trial insert 146 being used is too large, prior to inserting the trial insert 146 all of the way into the intervertebral space.

Consequently, the user may begin an operation to insert and position a rasp 102 in an intervertebral space by attaching various trial inserts 146 to the instrument 100. The user may then test the intervertebral gap to determine which size of rasp 102 is appropriate to use. The user may then detach the trial insert 146 from the instrument 100 and attach an appropriately sized rasp 102 to the distal end of the instrument 100.

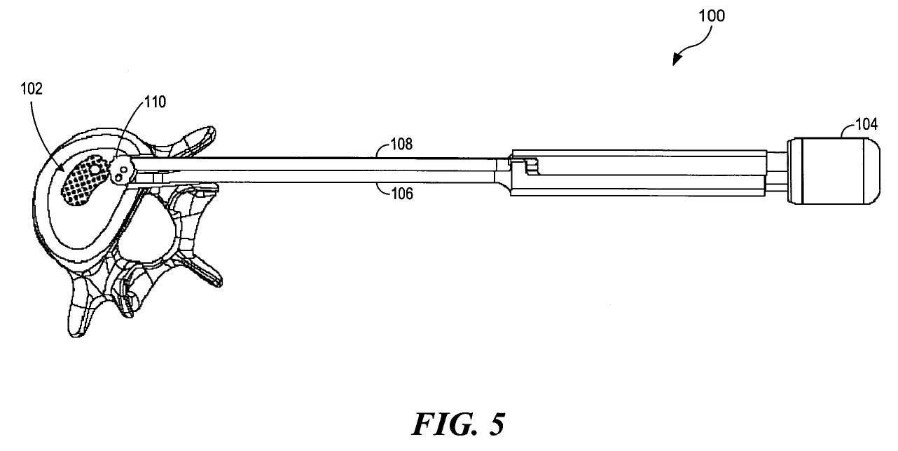

Generally, the user may rotate the rasp 102 to a straight orientation (i.e., designated by the angle “α1” in FIG. 2A) and introduce the rasp 102 to an area proximal to the intervertebral space. The user may then position the rasp 102 between, but outside of the adjacent vertebra and strike the instrument 100 on the knob 104. In some situations this action should insert the rasp 102 into the intervertebral space. The user may then rotate the rasp 102 to a rotated orientation (e.g., to the angle “α2”) using the knob 104. FIG. 5 illustrates the rasp 102 in the intervertebral space in a rotated position. If the user so desires, the user may then rotate the rasp 102 back and forth between the straight and rotated positions in order to decorticate the ends (or end plates) of the adjacent vertebrae. Once the user decides to withdraw the rasp 102 from the intervertebral space, the user may rotate the rasp 102 back to a straight position and withdraw the rasp 102 from the intervertebral space.

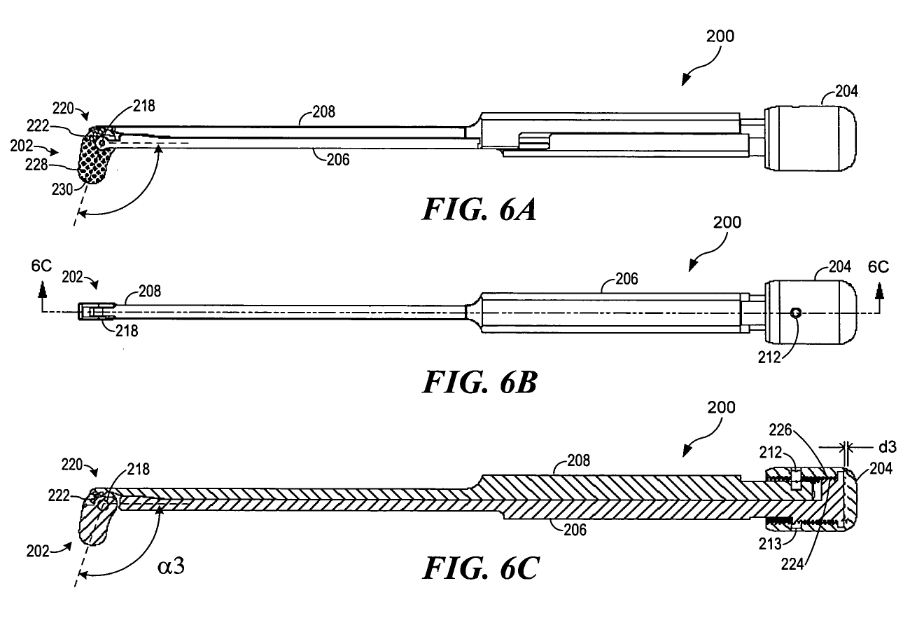

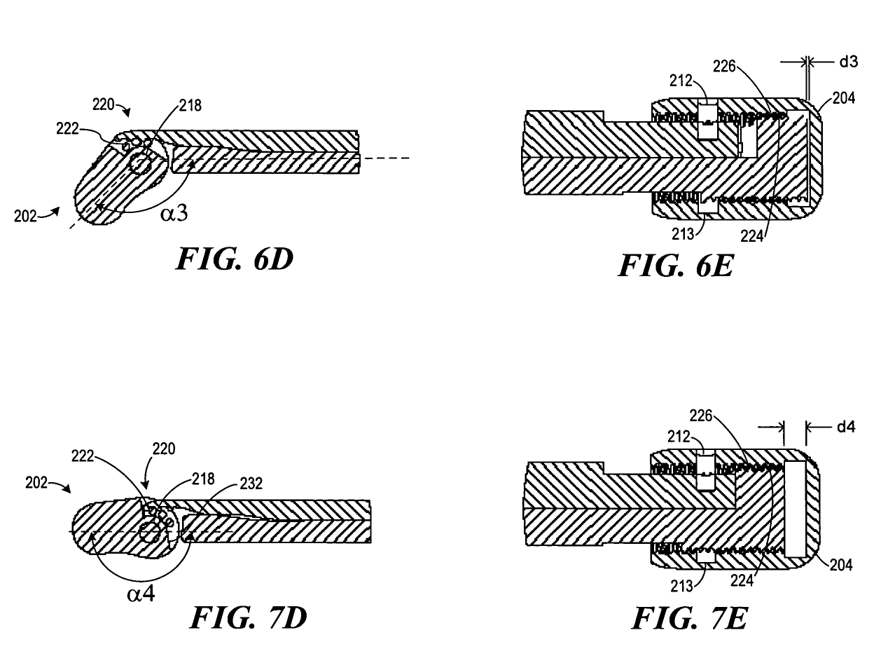

With reference now to FIGS. 6A to 10, another embodiment of the present invention is generally illustrated. More particularly, FIGS. 6A-6C illustrate an embodiment of an instrument 200 and a rasp 202 that are similar to an embodiment of the instrument 100 and rasp 102 previously disclosed. However, in certain embodiments, such as those illustrated in FIGS. 6A-6C for example, the instrument 200 and rasp 202 may differ from the instrument 100 and rasp 102 of other embodiments in the manner in which the rasp 202 is caused to rotate between a straight position and a rotated position.

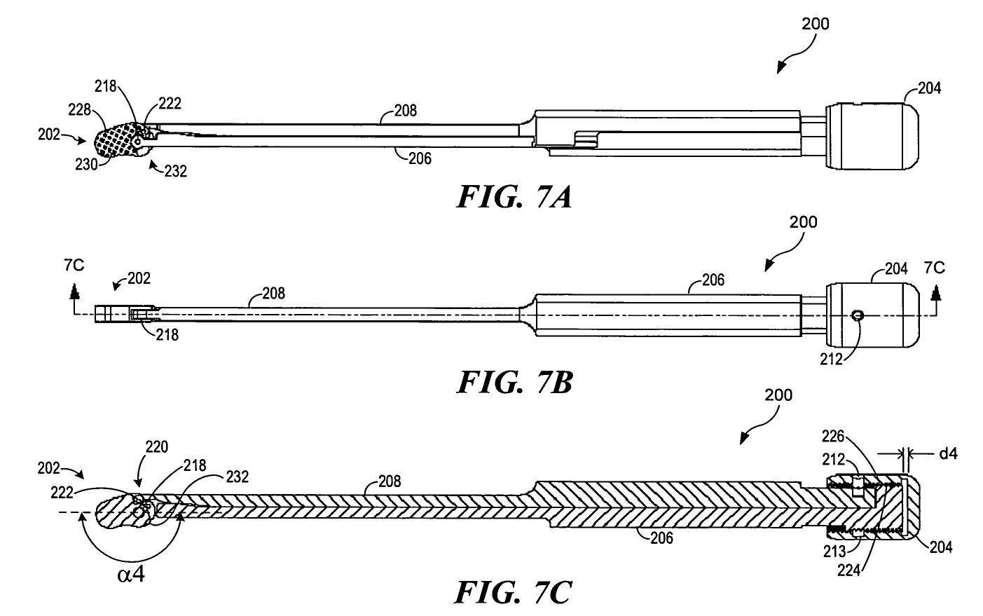

More particularly, the articulation bar 208 may comprise a set of gear teeth 220 or protrusions formed integrally on a surface of a distal end of the articulation bar 208, adjacent to an attached rasp 202. These gear teeth 220 may mesh with a series of pins 222 attached to the rasp 202. The series of pins 222 may be arranged along an arc on the rasp 202. Consequently, when the articulation bar 208 translates between positions respectively designated in FIGS. 6C and 7C as “d3” and “d4”, the rasp 202 may rotate between a rotated and a straight position, respectively designated by the angles “α3” and “α4”.

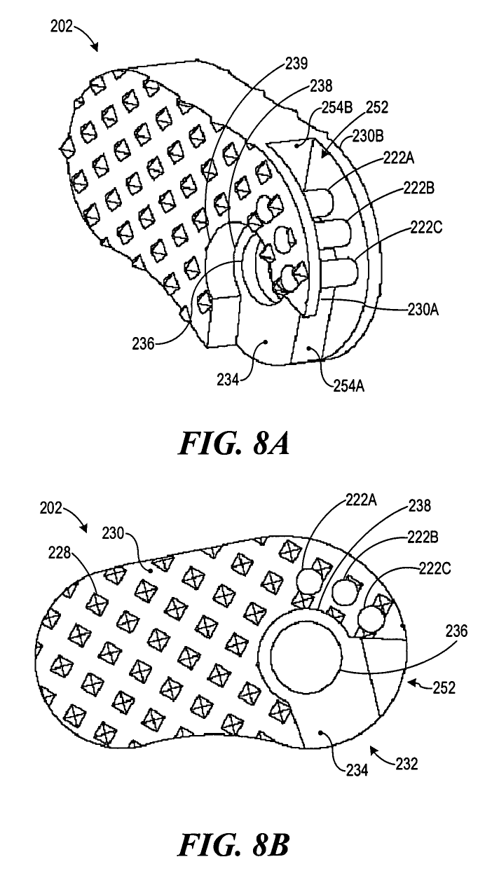

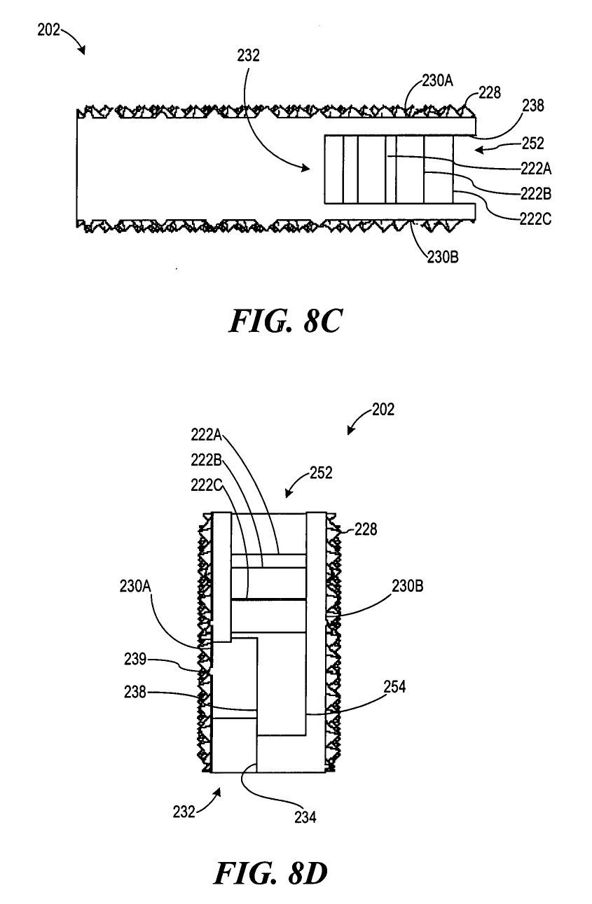

FIGS. 8A-8D illustrate further features of an embodiment of a rasp 202. More particularly, the rasp 202 may comprise an attachment area 232 at which location a pin 218 may pivotally couple the rasp 202 to the main body 206 via a hole 236. In addition, FIG. 8A illustrates that the rasp 202 may also comprise a rack and pinion type of arrangement for driving the rotation of the rasp 202. In some embodiments, such as the one shown in FIGS. 8A-8D for example, the rasp 202 may comprise a slot 252, which generally extends around a proximal end of the rasp 202. The extension of the slot 252 toward the attachment area 232 may provide the gear teeth 220 on the articulation bar 208 (which disengages from the pins 222) with sufficient clearance from portions of the rasp body 254 such that the rasp 202 may rotate at least until the last gear tooth 220 disengages from the last pin 222C. In the other direction, a similar extension of the slot 252 may enable the rasp 202 to rotate in the other direction until at least the first gear tooth 220 and the last pin 222A disengage. Alternatively, the rasp body surfaces 254A and 254B may be configured such that the surfaces 254A and 254B inhibit the articulation bar 208 from rotating beyond engagement with the pins 222A-C.

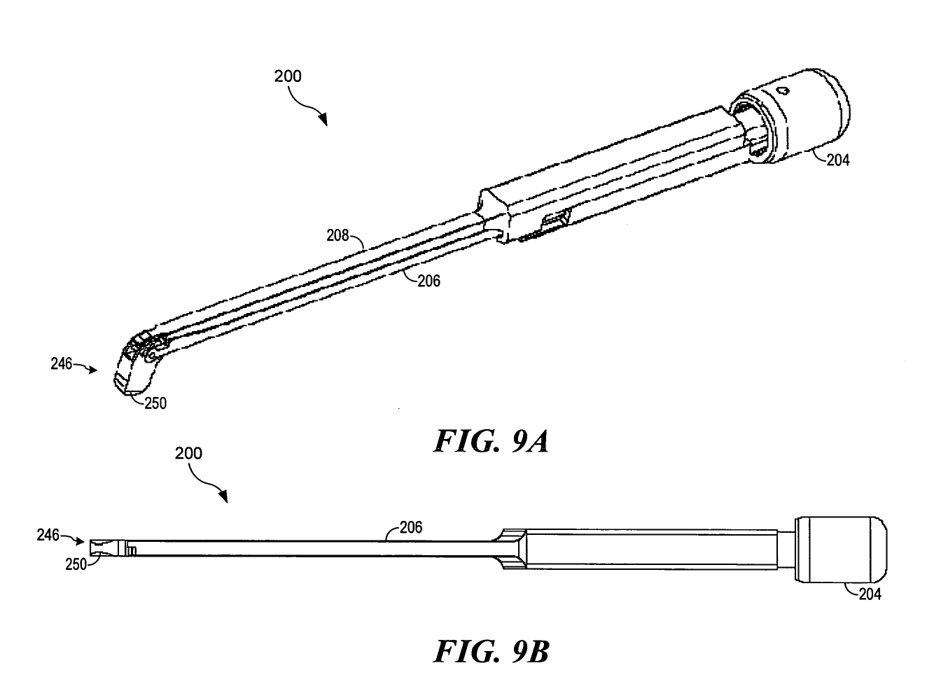

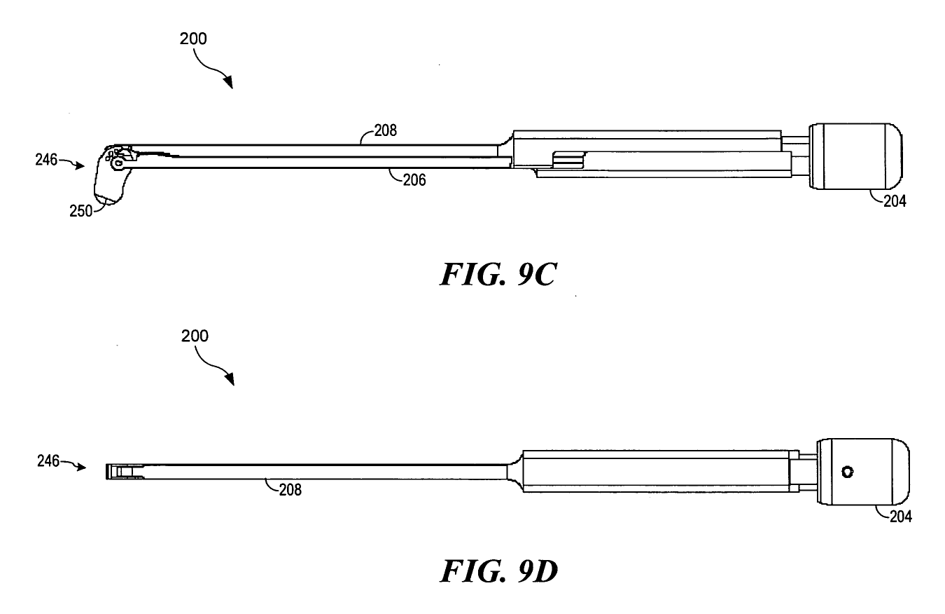

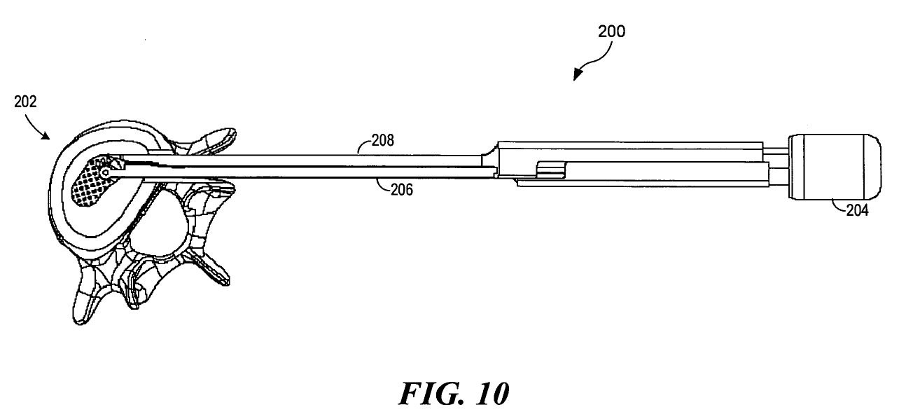

Furthermore, FIGS. 9A-9D illustrate that as with certain other embodiments, the instrument 200 may have a trial insert 246 removably attached to the instrument 200 (in a manner similar to the rasp 202) in order to determine an appropriate rasp 202 size. Therefore in some embodiments, as with the instrument 100 of certain other embodiments, the rasp 202 may also be removably attached to the instrument 200, inserted into the intervertebral space, rotated to decorticate the vertebra, and withdrawn from the intervertebral space via the instrument 200 (see FIG. 10).

The practice of an embodiment of the present invention may enable a decortication instrument to be inserted into an intervertebral space and used to prepare the vertebra for fusion in a minimally invasive manner. Moreover, because the rasps of certain embodiments rotate, the rasps may be used to decorticate areas that are otherwise difficult to reach because of obstructions. Likewise, since the rasps of certain embodiments are slightly larger (e.g., wider or longer) than the surgical spacers that a user may insert into an intervertebral space, the rasps may clean an area of the vertebra that is larger than the corresponding surface of the spacer (which may be inserted in the intervertebral space). Accordingly, the use of an embodiment of the present invention may encourage additional bone growth around the spacer, thereby creating better vertebral fusion. Further, the use of an embodiment of the present invention may improve the resulting bone growth and lead to the fusion of the vertebra and, ultimately, patient recovery. Moreover, the practice of an embodiment of the present invention may allow a user to determine an appropriate size of a rasp to use in a minimally invasive manner.

It is understood that the embodiments of the present invention can take many forms and configurations. Accordingly, several variations may be made in the foregoing without departing from the spirit or the scope of the subject matter of the invention. For example, with reference to FIG. 1F, any type of mechanism 10 (e.g., gears, sliders, electro-mechanical actuators) may be used to rotate the rasps 11 (and trial inserts) of some embodiments of the present invention when they are connected to an instrument 12.

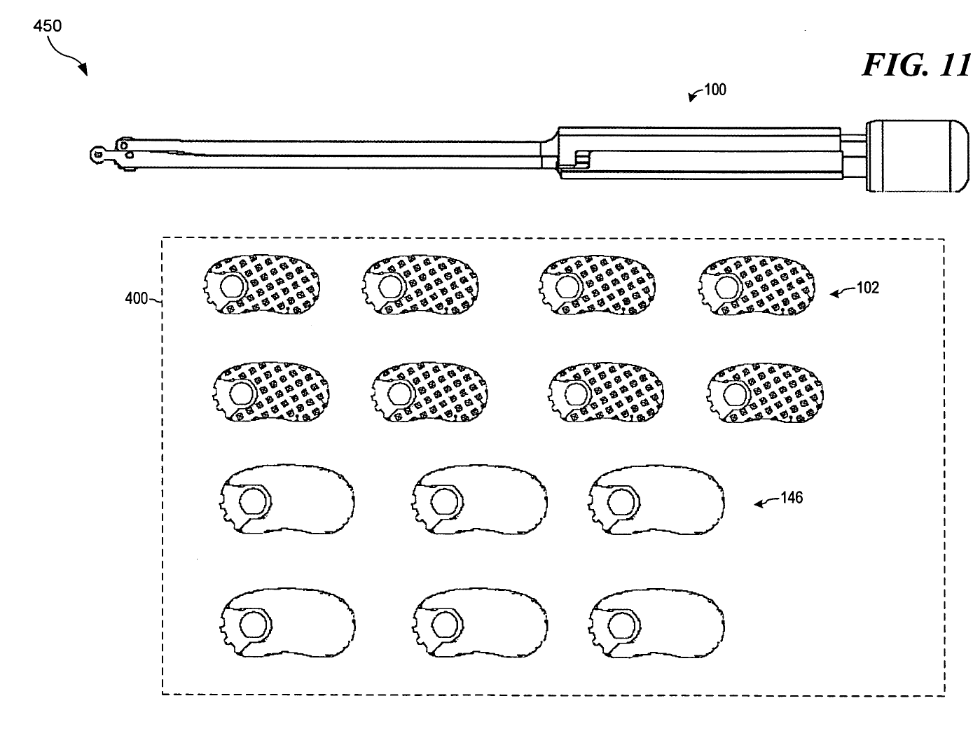

Turning now to FIG. 11, other illustrative embodiments of the present invention may include a surgical kit 450 that may comprise an instrument 100, and at least one rasp 102 and/or at least one trial insert 146. In the illustrative embodiment shown in the drawing, a plurality of rasps 102 and a plurality of trial inserts 146 may be contained within a storage device 400, such as a carrying case among others. The plurality of rasps 102 and the plurality of trial inserts 146 may differ from one another in one or more characteristic features. For example, the pluralities of rasps 102 and trail inserts 146 may have gradually changing thicknesses from one to another, either in predetermined increments and/or according to need. Other characteristic features may also be varied, for example, the plurality of rasps 102 may have gradually changing surface roughness from one rasp 102 to another. Additional or alternate features may also be altered as needed. The surgeon may then be able to use the plurality of trial inserts 146 to establish the distance between opposing surfaces of adjacent vertebrae and then select an appropriately sized rasp 102 from the plurality of rasps 102 to decorticate the boney surfaces.

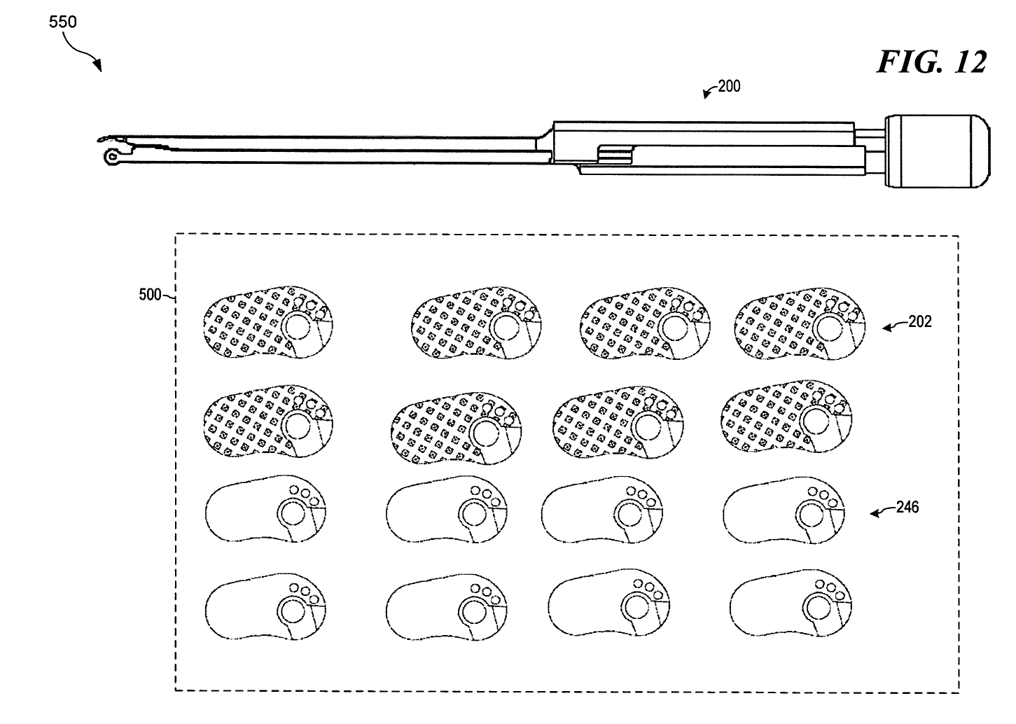

Referring now to FIG. 12, this drawing shows another illustrative embodiment a surgical kit 550. The surgical kit 550 that may comprise an instrument 200, and at least one rasp 202 and/or at least one trial insert 246. In the illustrative embodiment shown in the drawing, a plurality of rasps 202 and a plurality of trial inserts 246 may be contained within a storage device 500 such as a carrying case, among others. The plurality of rasps 202 and the plurality of trial inserts 246 may differ from one another in one or more characteristic features. For example, the pluralities of rasps 202 and trail inserts 246 may have gradually changing thicknesses from one to another, either in predetermined increments and/or according to need. Other characteristic features may also be varied, for example, the plurality of rasps 202 may have gradually changing surface roughness from one rasp 202 to another. Additional or alternate features may also be altered as needed. The surgeon may then be able to use the plurality of trial inserts 246 to establish the distance between opposing surfaces of adjacent vertebrae and then select an appropriately sized rasp 202 from the plurality of rasps 202 to decorticate the boney surfaces.

Other embodiments of a surgical instrument may include:

1. A surgical instrument that comprises:

a first member;

an attachment point on the first member configured to couple an insert to the instrument such that the insert is removable and configured to rotate relative to the instrument;

a mechanism coupled to the first member to pivot relative to the first member and comprising an arcuate portion configured to engage and rotate an arcuate end of an insert coupled to the first member such that the insert rotates relative to the arcuate portion of the mechanism;

a second member coupled to the first member for translation relative to the first member and coupled to the mechanism such that the mechanism is configured to pivot relative to the second member; and

an actuator coupled to the first member and to the second member such that movement of the actuator translates one of the first and the second members relative to the other of the first and the second members, thereby rotating the mechanism relative to the instrument.

2. The instrument of embodiment 1 further comprising a resilient member configured to couple the insert to the first member.

3. The instrument of embodiment 1 further comprising a threaded section configured to couple the actuator to one of the first and the second members such that movement of the actuator about the first and the second members translates one of the first and the second members relative to the other of the first and the second members.

4. The instrument of embodiment 3 further comprising a fixing member configured to substantially fix the actuator in position with regard to one of the first and the second members.

5. The instrument of embodiment 1 further comprising a plurality of recesses provided on the arcuate portion of the insert and a plurality of protrusions provided on the arcuate portion of the mechanism, the plurality of protrusions configured to correspond with the plurality of recesses such that the plurality of protrusions engage and rotate the insert coupled to the instrument.

6. The instrument of embodiment 1 wherein a rasp is coupled to the instrument as the insert.

7. The instrument of embodiment 1 wherein the insert comprises a first abutment surface and a second abutment surface for abutting opposing surfaces of an intervertebral space between a first and second vertebrae and configured such that the first abutment surface approaches the second abutment surface toward a distal end of the insert.

Still other embodiments of a surgical instrument may include:

1. A surgical instrument that comprises:

a first member;

an attachment point on the first member configured to couple an insert to the instrument such that the insert is removable and pivotal relative to the instrument;

a second member comprising at least one protrusion configured to engage and pivot a coupled insert; and

an actuator coupled to the first member and the second member, such that rotating the actuator about the first and the second members causes one of the first and the second members to move relative to the other, thereby pivoting a coupled insert relative to the instrument.

2. The instrument of embodiment 1 further comprising a resilient member configured to couple the insert to the first member such that the insert is configured to pivot relative to the first member.

3. The instrument of embodiment 1 further comprising a threaded section configured to couple the actuator to one of the first and the second members.

4. The instrument of embodiment 1 further comprising a fixing member configured to substantially fix the actuator in position with regard to one of the first and the second members.

5. The instrument of embodiment 1 further comprising a plurality of recesses provided on an insert and a plurality of protrusions provide on a distal end of the second member, the plurality of protrusions configured to correspond with the plurality of recesses such that the plurality of protrusions engage and rotate the insert coupled to the instrument.

6. The instrument of embodiment 1 further comprising a rack coupled to the second member and configured to engage a pinion coupled with the insert such that movement of the rack pivots the insert.

7. The instrument of embodiment 1 further comprising a plurality of gear protrusions coupled with the second member and configured to engage a plurality of pins coupled to an insert such that movement of the second member pivots the insert.

8. The instrument of embodiment 1 wherein a rasp is coupled to the instrument as the insert.

9. The instrument of embodiment 1 wherein the insert comprises a first abutment surface and a second abutment surface for abutting opposing surfaces of an intervertebral space between a first and second vertebrae and configured such that the first abutment surface approaches the second abutment surface toward a distal end of the insert.

Other embodiments of the method may include:

1. A method of decorticating a bone in vivo comprising:

coupling a temporary insert of a first thickness to an instrument such that the temporary insert is configured to be removable and pivotal;

attempting to insert the temporary insert into an intervertebral space;

replacing the temporary insert of the first thickness with a temporary insert of another thickness if insertion into the intervertebral space is inhibited or if substantial clearance exists between a boney surface of the intervertebral space and an opposing surface of the temporary insert of the first thickness;

repeating two previous steps as needed;

inserting the temporary insert into the intervertebral space and actuating a mechanism on the instrument configured to rotate the temporary insert relative to the instrument; and

withdrawing the temporary insert from the space using the instrument.

2. The method of embodiment 1 further comprising selecting the temporary insert from a plurality of trial inserts of various thicknesses.

3. The method of embodiment 1 further comprising selecting the temporary insert from a plurality of rasps of various thicknesses.

4. The method of embodiment 1 further comprising a resilient member configured to couple the temporary insert to the instrument such that the temporary insert is removable and pivotal relative to the instrument.

Other embodiments of the instrument may include:

1. A surgical instrument for spine surgery, comprising:

an elongated member having a length, a width, a proximal end portion and a distal end portion,

a guide member slidingly positioned adjacent to the elongated member, wherein the guide member has a length, a width, distal end portion and a proximal end portion;

a removable insert pivotably coupled to the distal end portion of the guide member, the removable insert having height between a top surface and an opposite bottom surface in a range between 4 mm and 20 mm, a distal end portion and an arcuate proximal end portion positioned between the top surface and the opposite bottom surface, wherein the arcuate proximal end portion includes a plurality of gear teeth;

at least one drive member pivotably coupled to the distal end portion of the elongated member, wherein the at least one drive member has a first partially circular surface with a plurality of projections that slippingly engage the plurality of gear teeth of the removable insert;

an actuating mechanism coupled to the proximal end portions of the elongated member and the guide member configured to move the elongated member relative to the guide member; and

a handle inline with the actuator mechanism and threadingly coupled to the guide member or the elongated member, the handle having an impaction surface with a width that is greater than the width of the elongated member or the width of the guide member.

2. The instrument of embodiment 1 wherein the top surface of the removable insert is generally smooth.

3. The instrument of embodiment 1 wherein the top surface of the removable insert has a plurality of teeth.

4. The instrument of embodiment 1 wherein the top surface and the opposite bottom surface of the distal end portion of the removable insert is tapered toward each other.

5. The instrument of embodiment 1 further comprising a locking member coupled to the handle, and coupled to the elongated member or the guide member.

Other embodiments of the instrument may include:

1. A surgical instrument for spine surgery, comprising:

an elongated member having a length, a width, a proximal end portion and a distal end portion,

a guide member slidingly positioned adjacent to the elongated member, wherein the guide member has a length, a width, distal end portion and a proximal end portion;

a removable insert pivotably coupled to the distal end portion of the guide member, the removable insert having a top surface, an opposite bottom surface, a distal end portion and an arcuate proximal end portion positioned between the top surface and bottom surface, wherein the arcuate proximal end portion includes a plurality of pins extending between the top surface and the opposite bottom surface;

a pawl member that extends along a curved longitudinal axis and is integral to the distal end portion of the elongated member, wherein the pawl member slippingly engages the plurality of pins of the removable insert;

an actuating mechanism coupled to the proximal end portions of the elongated member and the guide member configured to move the elongated member relative to the guide member; and

a handle coupled to the actuator mechanism and having an impaction surface with a width that is greater than the width of the elongated member or the width of the guide member.

2. The instrument of embodiment 1 wherein the top surface of the removable insert is generally smooth.

3. The instrument of embodiment 1 wherein the top surface of the removable insert has a plurality of teeth.

4. The instrument of embodiment 1 wherein the top surface and the opposite bottom surface of the distal end portion of the removable insert taper toward each other.

Still other embodiments of the instrument may include:

1. A surgical instrument for spine surgery, comprising:

an elongated member having a length, a width, a proximal end portion and a distal end portion,

a guide member slidingly positioned adjacent to the elongated member, wherein the guide member has a length, a width, distal end portion and a proximal end portion;

an insert pivotably coupled to the distal end portion of the guide member, the insert having a top surface, an opposite bottom surface, a distal end portion and an arcuate proximal end portion positioned between the top surface and the opposite bottom surface, wherein the arcuate proximal end portion includes a plurality of engagement members;

at least one drive member coupled to the distal end portion of the elongated member, wherein the at least one drive member has a first surface that slippingly engages the plurality of engagement members of the a removable insert; and

an actuating mechanism coupled to the proximal end portions of the elongated member and the guide member configured to move the elongated member relative to the guide member.

2. The instrument of embodiment 1 wherein the engagement members are a plurality of gear teeth.

3. The instrument of embodiment 1 wherein the engagement members are a plurality of pins extending between the top surface and the opposite bottom surface.

4. The instrument of embodiment 1 wherein the first surface of the guide member is generally circular.

5. The instrument of embodiment 1 further comprising a plurality of projections located on the first surface of the elongated member.

6. The instrument of embodiment 1 wherein the first surface of the elongated member extends along curved longitudinal axis.

7. The instrument of embodiment 1 further comprising a plurality of projections located on the first surface of the elongated member.

8. The instrument of embodiment 1 further comprising a handle coupled to the actuator mechanism.

9. The instrument of embodiment 7 wherein the handle has an impaction surface having a diameter greater than the width of the elongated member or the guide member.

10. The instrument of embodiment 1 wherein the top surface of the removable insert is generally smooth.

11. The instrument of embodiment 1 wherein the top surface of the removable insert has a plurality of teeth.

Still further embodiments of the instrument may include:

1. A surgical instrument for spine surgery, comprising:

an elongated member having a length, a width, a proximal end portion and a distal end portion,

a guide member slidingly positioned adjacent to the elongated member, wherein the guide member has a length, a width, distal end portion and a proximal end portion;

an insert pivotably coupled to the distal end portion of the guide member, the insert having height between a top surface and an opposite bottom surface in a range between 4 mm and 20 mm, a distal end portion and an arcuate proximal end portion positioned between the top surface and the opposite bottom surface, wherein the arcuate proximal end portion includes a plurality of engagement members;

at least one drive member coupled to the distal end portion of the elongated member, wherein the drive member has a first generally arcuate surface that slippingly engages the plurality of engagement members of the insert; and

an actuating mechanism coupled to the proximal end portions of the elongated member and the guide member configured to move the guide member relative to the elongated member.

2. The instrument of embodiment 1 wherein the engagement members are a plurality of gear teeth.

3. The instrument of embodiment 1 wherein the engagement members are a plurality of pins extending between the top surface and the opposite bottom surface.

4. The instrument of embodiment 1 further comprising a plurality of projections located on the first surface of the drive member.

5. The instrument of embodiment 1 further comprising a handle coupled to the actuator mechanism.

6. The instrument of embodiment 5 wherein the handle has an impaction surface having a width greater than the width of the elongated member or the width of the guide member.

7. The instrument of embodiment 5 wherein the handle has a domed impaction surface.

Still more embodiments of the instrument may include:

1. A surgical instrument for spine surgery, comprising:

a guide member having a proximal end portion and a distal end portion;

an insert member having a means for removably attaching to the distal end portion of the guide member, the insert having a top surface and a bottom surface;

an elongated member slidingly positioned adjacent to the guide member, the elongated member having a means for pivoting the insert member from a first position relative to the elongated member to a second position relative to the elongated member;

an actuating mechanism having a means for translating the elongated member relative to the guide member and a means for locking the first position and the second position of the insert; and

a handle in line with the actuating mechanism having a means for absorbing and transferring an impaction force to the insert member.

2. The surgical instrument of embodiment 1 wherein the top surface and the bottom surface of the insert member have a means for traumatizing bone.

3. The surgical instrument of embodiment 2 wherein the means for traumatizing bone includes a plurality of teeth.

4. The surgical instrument of claim 2 wherein the means for traumatizing bone includes a textured surface.

5. The surgical instrument of claim 1 wherein the top surface and the bottom surface of the insert member are generally smooth.

Still other embodiments of the method may include:

1. A method of traumatizing a pair of adjacent vertebral endplates comprising:

providing a surgical instrument having a pivoting distal insert, a proximal handle portion and a body portion positioned between the distal insert and the proximal handle portion, the distal insert having a first angular position relative to the body and the distal insert having a textured top and bottom surfaces;

placing a leading end of the distal insert in a first position between two adjacent vertebral endplates;

moving the distal insert to a second position between the adjacent vertebral endplates by impacting the proximal end portion of the surgical instrument;

pivoting the distal insert to a second angular position relative to the body by rotating the handle about the body;

locking the second angular position of the distal insert; and

moving the distal insert to a third position between the adjacent vertebral endplates by impacting the proximal end portion of the surgical instrument.

2. The method of claim 1 further comprising the steps of:

removing the distal insert from between the adjacent vertebral endplates;

detaching the distal insert from the surgical instrument; and

replacing the distal insert with a second distal insert chosen from a kit having a plurality of distal inserts.

3. The method claim 2 wherein the plurality of distal inserts have

a height between the top and the bottom surfaces in a range between 4 mm and 20 mm.

4. The method of claim 3 further comprising the steps of:

placing a leading end of the second distal insert in a first position between two adjacent vertebral endplates;

moving the second distal insert to a second position between the adjacent vertebral endplates by impacting the proximal end portion of the surgical instrument;

pivoting the second distal insert to a second angular position relative to the body by rotating the handle about the body;

locking the second angular position of the second distal insert; and

moving the second distal insert to a third position between the adjacent vertebral endplates by impacting the proximal end portion of the surgical instrument.

Having thus described various aspects of the present invention by reference to certain exemplary embodiments, it is noted that the embodiments disclosed are illustrative rather than limiting in nature. A wide range of variations, modifications, changes, and substitutions are contemplated in the foregoing disclosure. In some instances, some features of embodiments of the present invention may be employed without a corresponding use of other features. Many such variations and modifications may be considered obvious and desirable by those skilled in the art based upon a review of the foregoing description of the illustrative embodiments. Accordingly, it is appropriate that the appended claims be construed broadly and in a manner consistent with the scope of the invention.

This invention claims:

1. A surgical instrument for traumatizing a pair of vertebral endplates, the surgical instrument comprising:
an elongated member having a length, a width, a proximal end portion and a distal end portion,
a guide member positioned adjacent to the elongated member, wherein the guide member has a length, a width, distal end portion and a proximal end portion;
a removable insert pivotably coupled to the distal end portion of the guide member, the removable insert having height between a top surface and an opposite bottom surface, a distal end portion and an arcuate proximal end portion positioned between the top surface and the opposite bottom surface, wherein the arcuate proximal end portion includes a plurality of gear teeth;
at least one drive member pivotably coupled to the distal end portion of the elongated member, wherein the at least one drive member has a first partially circular surface with a plurality of projections that slippingly engage the plurality of gear teeth of the removable insert;
an actuating mechanism coupled to the proximal end portions of the elongated member and the guide member configured to move the elongated member relative to the guide member; and
a handle coupled to the actuator mechanism and threadingly coupled to the guide member or the elongated member, the handle having an impaction surface.

2. The instrument of claim 1 wherein the top surface of the removable insert is generally smooth.

3. The instrument of claim 1 wherein the top surface of the removable insert has a plurality of teeth.

4. The instrument of claim 1, wherein the height is in a range between 4 mm and 20 mm.

5. The instrument of claim 1 further comprising a locking member coupled to the handle, and coupled to the elongated member or the guide member.

6. A surgical instrument for spine surgery, comprising:
an elongated member having a length, a width, a proximal end portion and a distal end portion,
a guide member slidingly positioned adjacent to the elongated member, wherein the guide member has a length, a width, distal end portion and a proximal end portion;
a removable insert pivotably coupled to the distal end portion of the guide member, the removable insert having a top surface, an opposite bottom surface, a distal end portion and an arcuate proximal end portion positioned between the top surface and bottom surface, wherein the arcuate proximal end portion includes a plurality of pins extending between the top surface and the opposite bottom surface;
a pawl member that extends along a curved longitudinal axis and is integral to the distal end portion of the elongated member, wherein the pawl member slippingly engages the plurality of pins of the removable insert;
an actuating mechanism coupled to the proximal end portions of the elongated member and the guide member configured to move the elongated member relative to the guide member; and
a handle coupled to the actuator mechanism and having an impaction surface with a width that is greater than the width of the elongated member or the width of the guide member.

7. The instrument of claim 6 wherein the top surface of the removable insert is generally smooth.

8. The instrument of claim 6 wherein the top surface of the removable insert has a plurality of teeth.

9. A surgical instrument for spine distraction, comprising:
an elongated member having a length, a width, a proximal end portion and a distal end portion,
a guide member positioned adjacent to the elongated member, wherein the guide member has a length, a width, distal end portion and a proximal end portion;
an insert pivotably coupled to the distal end portion of the guide member, the insert having a top surface, an opposite bottom surface, a distal end portion and an arcuate proximal end portion positioned between the top surface and the opposite bottom surface, wherein the arcuate proximal end portion includes a plurality of engagement members;
at least one drive member coupled to the distal end portion of the elongated member, wherein the at least one drive member has a first surface that slippingly engages the plurality of engagement members of the a removable insert; and
an actuating mechanism coupled to the proximal end portions of the elongated member and the guide member configured to move the elongated member relative to the guide member.

10. The instrument of claim 9 wherein the engagement members are a plurality of pins extending between the top surface and the opposite bottom surface.

11. The instrument of claim 9 further comprising a plurality of projections located on the first surface of the elongated member.

12. The instrument of claim 9 wherein the first surface of the elongated member extends along curved longitudinal axis.

13. The instrument of claim 9 further comprising a plurality of projections located on the first surface of the elongated member.

14. The instrument of claim 9 further comprising a handle coupled to the actuator mechanism.

15. A surgical instrument for spine surgery, comprising:
an elongated member having a length, a width, a proximal end portion and a distal end portion,
a guide member slidingly positioned adjacent to the elongated member, wherein the guide member has a length, a width, distal end portion and a proximal end portion;
an insert pivotably coupled to the distal end portion of the guide member, the insert having height between a top surface and an opposite bottom, a distal end portion and an arcuate proximal end portion positioned between the top surface and the opposite bottom surface, wherein the arcuate proximal end portion includes a plurality of engagement members;
at least one drive member coupled to the distal end portion of the elongated member, wherein the drive member has a first generally arcuate surface that slippingly engages the plurality of engagement members of the insert; and
an actuating mechanism coupled to the proximal end portions of the elongated member and the guide member configured to move the guide member relative to the elongated member.

16. The instrument of claim 15 wherein the engagement members are a plurality of gear teeth.

17. The instrument of claim 15 wherein the engagement members are a plurality of pins extending between the top surface and the opposite bottom surface.

18. The instrument of claim 15 further comprising a plurality of projections located on the first surface of the drive member.

19. The instrument of claim 15 further comprising a handle coupled to the actuator mechanism.

20. A surgical kit for spine surgery, comprising:
an insertion instrument, the instrument comprising:
a guide member having a proximal end portion and a distal end portion;
an elongated member slidingly positioned adjacent to the guide member, the elongated member having a means for pivoting the insert member from a first position to a second position;
an actuating means for translating the elongated member relative to the guide member,
a means for locking the first position and the second position of the insert; and
a handle means coupled to the actuating means having a means for absorbing and transferring an impaction force to the insert member,
a plurality of insert members, each having a means for removably attaching to the distal end portion of the guide member, wherein each insert has a top surface and a bottom surface, and a different height between the top surface and the bottom surface.

21. The surgical kit of claim 20 wherein the top surface and the bottom surface of the insert members have a means for traumatizing bone.

22. A method of traumatizing a pair of adjacent vertebral endplates comprising:
providing a surgical instrument having a pivoting distal insert, a proximal handle portion and a body portion positioned between the distal insert and the proximal handle portion, the distal insert having a first angular position relative to the body and the distal insert having a textured top and bottom surfaces;
placing a leading end of the distal insert in a first position between two adjacent vertebral endplates;
moving the distal insert to a second position between the adjacent vertebral endplates by impacting the proximal end portion of the surgical instrument;
pivoting the distal insert to a second angular position relative to the body by rotating the handle about the body;
locking the second angular position of the distal insert; and
moving the distal insert to a third position between the adjacent vertebral endplates by impacting the proximal end portion of the surgical instrument.

23. The method of claim 22 further comprising the steps of:
removing the distal insert from between the adjacent vertebral endplates;
detaching the distal insert from the surgical instrument; and
replacing the distal insert with a second distal insert chosen from a kit having a plurality of distal inserts.

24. The method of claim 22 further comprising the steps of:
placing a leading end of the second distal insert in a first position between two adjacent vertebral endplates;
moving the second distal insert to a second position between the adjacent vertebral endplates by impacting the proximal end portion of the surgical instrument;
pivoting the second distal insert to a second angular position relative to the body by rotating the handle about the body;
locking the second angular position of the second distal insert; and
moving the second distal insert to a third position between the adjacent vertebral endplates by impacting the proximal end portion of the surgical instrument.
